(19) United States
(12) Patent Application Publication
(10) Pub. No.: US 2022/0323786 A1
(43) Pub. Date: Oct. 13, 2022

(54) APPARATUSES AND METHODS FOR TREATING PERIIMPLANTITIS USING UVC

(71) Applicant: BIOLASE, INC., Foothill Ranch, CA (US)

(72) Inventors: Robert J. Miller, Delray Beach, FL (US); William E. Brown, Tuscaloosa, AL (US)

(21) Appl. No.: 17/716,904

(22) Filed: Apr. 8, 2022

Related U.S. Application Data
(60) Provisional application No. 63/172,486, filed on Apr. 8, 2021.

(51) Int. Cl. A61N 5/06; A61C 8/02; A61N 5/067; A61C 1/14; A61C 1/00

(52) U.S. Cl. CPC A61N 5/0624; A61C 8/0006; A61N 5/067; A61C 1/147; A61C 1/141; A61C 1/0046; A61N 2005/0606; A61N 2005/0661

(57) ABSTRACT

The disclosure is directed to systems and methods for treatment of periimplantitis using ultraviolet light. In some embodiments, the ultraviolet light is ultraviolet c light with a wavelength between 100 and 280 nanometers. In some embodiments, the ultraviolet c light makes the surface of the implant hydrophilic and more receptive to cell protein attachment. In some embodiments, an apparatus for treating periimplantitis using ultraviolet light includes a handpiece with a flexible neck and a rotatable tip at the end of the flexible neck. In some embodiments, the handpiece includes a contra angle dental handpiece or a straight dental handpiece which can couple to and direct ultraviolet light from either an end firing tip or a side firing tip. In some embodiments, the handpiece is sized to enable UVC light to be directed to an exposed area of an implant facing toward one or more posterior portions of the oral cavity.

### RELATED APPLICATION

This application claims the benefit and priority of U.S. Provisional Patent Application No. 63/172,486, filed Apr. 8, 2021, entitled “TREATMENT OF PERI-IMPLANTITIS USING ULTRAVIOLET-C LASERS,” which is incorporated herein by reference in its entirety.

### BACKGROUND

Placing dental implants is an increasingly important service which can significantly improve a patient's health and overall quality of life. It is important that surrounding soft tissue and bone osseointegrate with an implant. Recently, it has been discovered that certain methods of treating the surface of implants (e.g., titanium, zirconium) with UVC (Ultraviolet C) light or Non-Thermal Plasma (“NTP”) before they are initially implanted improves hydrophilic properties of the implants. This results in fluids and proteins from surrounding cells being drawn to the surface of the implant to form a more secure bond. Current methods of treating implants with UVC light are limited to placing new implants in sealed containers which include UVC lamps and then exposing the implants to intense UVC light from 10 seconds to 15 minutes or more. However, there is currently no apparatus or method for in-situ treatment of the exposed surfaces of embedded implants which have partially or completely failed to bond to surrounding tissue.

Patients that develop peri-mucositis and peri-implantitis over time due to infection, an adverse body reaction to dental materials, and/or bone loss at the implant site require revision surgery. In these cases, degeneration of the bone leaves little to nothing for the implant screws to hold on to, so they are in effect “free floating” at the implant site. While these free-floating implants can be easily removed, removal is undesirable for implants that are still at least partially attached to bone. In the current state of the art, there is only about a 50% success rate in achieving the same hydrophilic bond with new tissue and graft material on implants as is seen in new implants. These unsuccessful cases become refractory, meaning after an implant repair procedure, many cases relapse.

Therefore, there is a need for a method and apparatus that significantly improve the success rates for revision surgery implants.

### SUMMARY

In some embodiments, the disclosure is directed to an apparatus and method for treating implants embedded in a patient's mouth. In some embodiments, the method includes one or more of the following steps: (1) cleaning the area by removing granulation tissue and/or pathologic tissue from the defect area; (2) preparing the area for graft material; (3) removing remaining bioburden and/or treat the exposed implant surfaces with directed laser energy (e.g., from an Er, Cr:YSGG 2790 nm Laser, a 9300 or 10.600 nm CO2 laser, a 1064 nm Nd:YAG or NIR diode laser; or an Er:YAG 2940 nm laser); (4) applying a UVC treatment to the exposed implant surfaces using an instrument; (5) applying a graft material to rebuild the peri-implantitis affected area; and (6) applying a collagen barrier, which secures the implant at the same location for healing and restoration.

In some embodiments, the disclosure is also directed to an ultraviolet (UV) instrument configured and arranged to deliver ultraviolet light such as UVC light at 100-400 nm to the exposed implant surfaces. While the specific wavelength UVC is used in connection with some embodiments throughout this disclosure, it is understood that any disclosure of a method and/or apparatus that uses UVC (e.g., the 100-280 nm portion of the UV spectrum) is also a disclosure of a method and/or apparatus that uses of ultraviolet light in a wavelength of 100-400 nm, or an apparatus that emits plasma from one or more tip openings. In some embodiments, the disclosure is also directed to a UVC instrument configured and arranged to deliver UVC light at 253.7 nm to the exposed implant surfaces. In some embodiments, the disclosure is directed to an NTP instrument configured and arranged to deliver a plasma photofunctionalization process to the exposed implant surfaces. In some embodiments, the photofunctionalization process includes emitting hot or cold plasma energy from a tip opening of the instrument. FIG. 12 serves as a reference to the shape and configuration of both a plasma instrument and/or a UVC instrument according to some embodiments. As used herein, an exposed implant surface is any portion of an implant of any material that is surgically secured within the mouth of a patient, and where at least a portion of the implant is not covered by bone and/or tissue during a treatment procedure. Some embodiments of the system are directed to a UVC instrument and directional delivery system instrument configured and arranged to apply UVC light at 253.7 nm to implant surfaces including a posterior portion of an exposed implant surface.

In some embodiments, the UVC instrument comprises one or more fluid tubes. In some embodiments, at least one of the one or more fluid tubes is a fluid delivery tube configured and arranged to deliver a fluid to or adjacent the implant site. In some embodiments, the fluid includes water. In some embodiments, the fluid includes an acid etch. In some embodiments, the fluid includes hydrogen peroxide. In some embodiments, the fluid is a gas. In some embodiments, the fluid is air.

In some embodiments, at least one of the one or more fluid tubes are fluid cooling tubes configured to remove heat from the UVC instrument. In some embodiments, the one or more fluid tubes are configured and arranged to circulate or move cooling fluid into and out of a UVC tip comprising at least a portion of a UVC delivery system. In some embodiments, at least a portion of the UVC instrument adjacent to the UVC tip is a neck or other structure that is flexible and/or adjustable. In some embodiments, the neck can bend up to 180° relative to the handpiece such that UVC light can be directed onto an implant surface facing the posterior portions of the oral cavity while the instrument handpiece extends outside the patient's mouth.

In some embodiments, the UVC instrument includes one or more UVC tips. In some embodiments, the one or more UVC tips are removable. In some embodiments, at least a portion of the one or more UVC tips includes a light opening configured and arranged to allow UVC light to pass therethrough. In some embodiments, the opening is at a distal end of the UVC tip and is positioned to where a plane parallel to the opening at the surface and parallel to the distal surface would be substantially perpendicular to a plane bisecting a central axis of the handpiece and/or the neck, such that the UVC light is directed from the distal end onto the implant surface. In some embodiments, the light opening is located on a side of the one or more UVC tips adjacent to the distal end of the tip to where a plane tangent and/or parallel to the opening and orthogonal to the distal end positioned along a side surface of the tip would be parallel to a plane bisecting a central axis of the handpiece and/or the neck, such that the UVC light is directed from a side surface of the tip onto the implant surface. In some embodiments, at least a portion of the one or more UVC tips that include a UVC opening on the side is rotatable such that the UVC light can be directed at any angle from 0° to 360°.

### DRAWING DESCRIPTION

FIG. 1 depicts the mechanism for the transformation of an implant surface from hydrophilic to hydrophobic according to some embodiments.

FIG. 2 is a depiction of cell affinity for acid-etched titanium surfaces with different ages and with or without ultraviolet treatment according to some embodiments.

FIG. 3 is an illustration of how cell proteins react with a UVC treated implant surface according to some embodiments.

FIG. 4 illustrates the complex fibrin attachment structure after a UVC treatment according to some embodiments.

FIG. 5 depicts an undesirable attachment structure for an implant not treated with the UVC treatment described herein according to some embodiments.

FIG. 6 is a radiograph of an example peri-implantitis site before treatment according to the methods described herein according to some embodiments.

FIG. 7 is a high-resolution picture of the peri-implantitis site shown in FIG. 6 before treatment according to the methods described herein according to some embodiments.

FIG. 8 is a picture of an ablative laser suitable for the methods described herein according to some embodiments.

FIG. 9 is a high-resolution picture showing the ablative laser cleaning the exposed implant surfaces according to some embodiments.

FIG. 10 depicts an acid etching step in accordance with some embodiments.

FIG. 11 shows a UVC instrument that utilizes direct exposure from a mercury lamp generating UVC at 254 nm according to some embodiments.

FIG. 12 illustrates a handpiece comprising a flexible neck and interchangeable tips for directing ultraviolet light (e.g., UVC light) onto a partially exposed implant according to some embodiments.

FIG. 13 illustrates two interchangeable handpiece variations and removable light transmission component according to some embodiments.

FIG. 14 depicts a straight handpiece with one or more UVC light emitting diodes (LEDs) at a distal end according to some embodiments.

FIG. 15 shows a contra-angle handpiece and one or more UVC light projecting arrangements according to some embodiments.

FIG. 16 illustrates a straight handpiece and light projecting arrangement according to some embodiments.

FIG. 17 shows the insertion of grafting material into the implant site according to some embodiments.

FIG. 18 shows a three-month post-op radiograph showing a successful bond of the graft material to the remaining implants according to some embodiments.

FIG. 19 illustrates a computer system enabling or comprising the systems and methods in accordance with some embodiments of the system.

### DETAILED DESCRIPTION

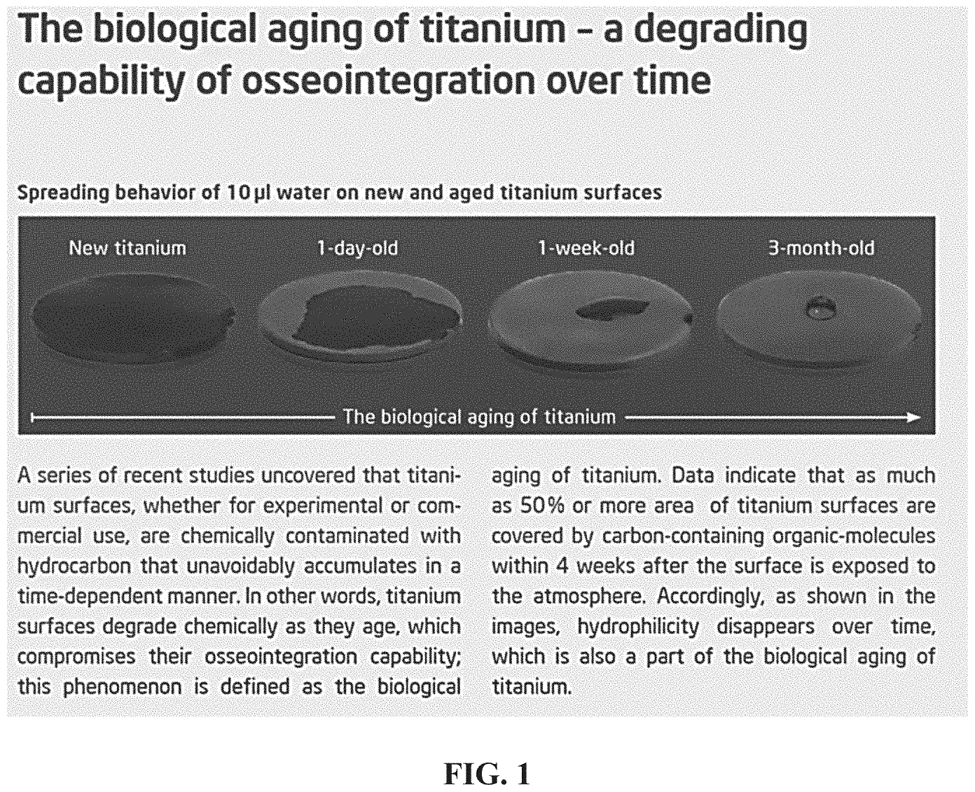

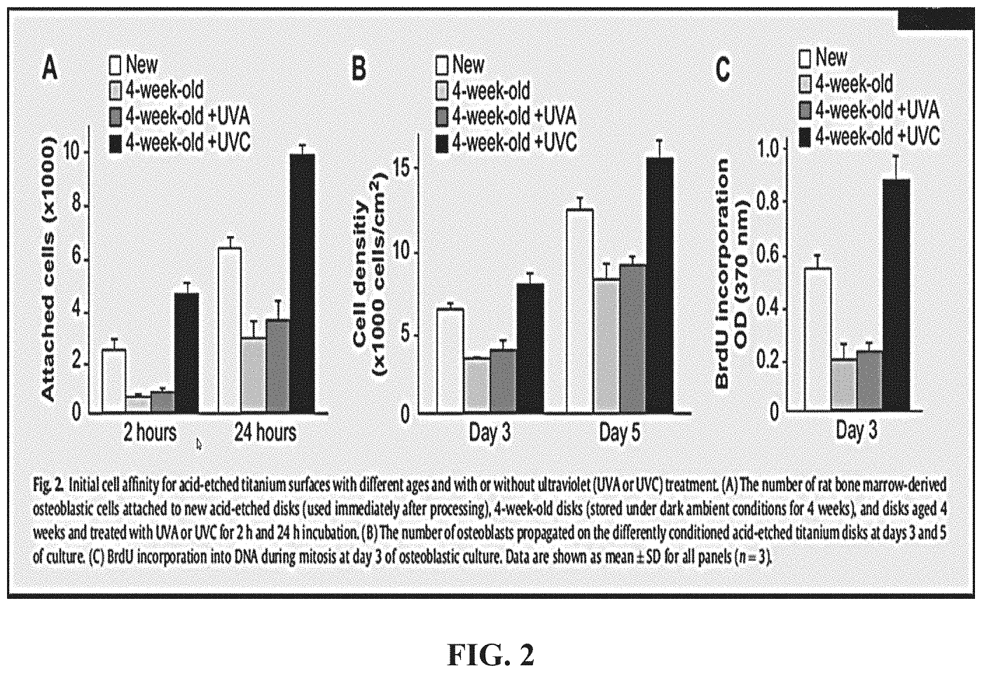

FIG. 1 depicts the mechanism for the transformation of an implant surface from hydrophilic to hydrophobic according to some embodiment. As titanium ages, exposed surfaces become contaminated with carbon-containing organic molecules as well as other non-organic molecules present in the atmosphere. The reduction in hydrophilicity caused by the contamination causes fluid repulsion. As shown in FIG. 2, this repulsion effect also applies to the fluids in cells. However, studies have shown that after surface treatment with ultraviolet light, in particular UVC light, cell affinity dramatically increases.

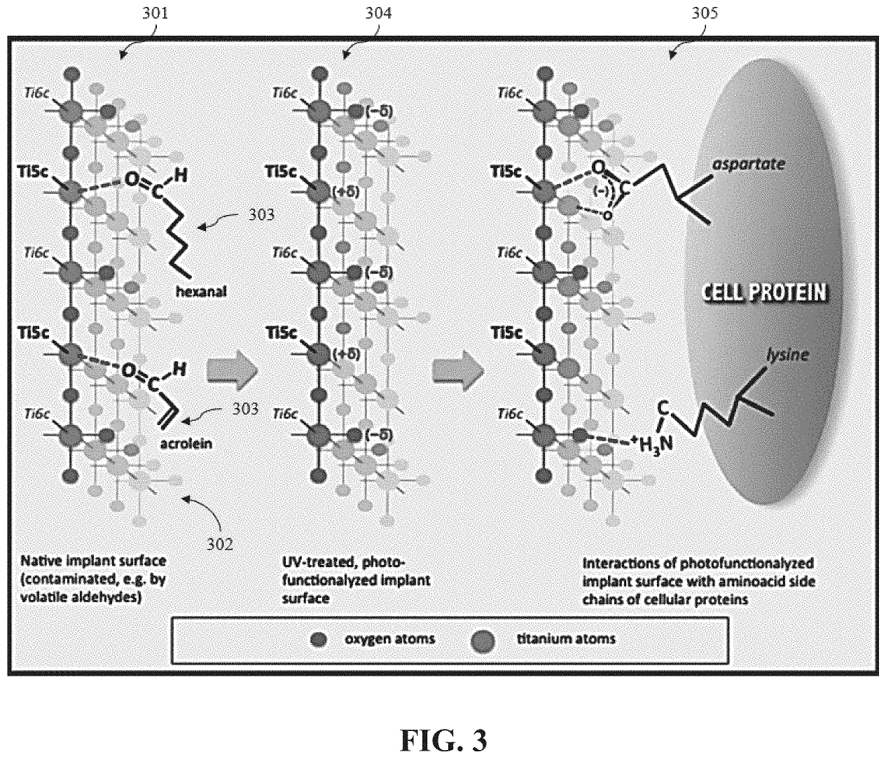

FIG. 3 is an illustration showing how cell proteins react with a UVC treated implant surface. As shown in the far-left column 301, organic and/or inorganic materials from the atmosphere bind to the implant 302 through Van der Waals forces to form surface contaminants 303. This binding not only contaminants the surface, but also changes the surface's electron state. The middle column 304 shows how the application of UVC at approximately 254 nm removes the surface contaminants 303 which also changes the electron state. This in turn makes the surface of the implant hydrophilic and receptive to cell protein attachment as shown in the right column 305. The implant is now receptive to the attachment of cells, and bodily fluid (e.g., blood) will wick up its surface as the systems and methods described herein are implemented according to some embodiments.

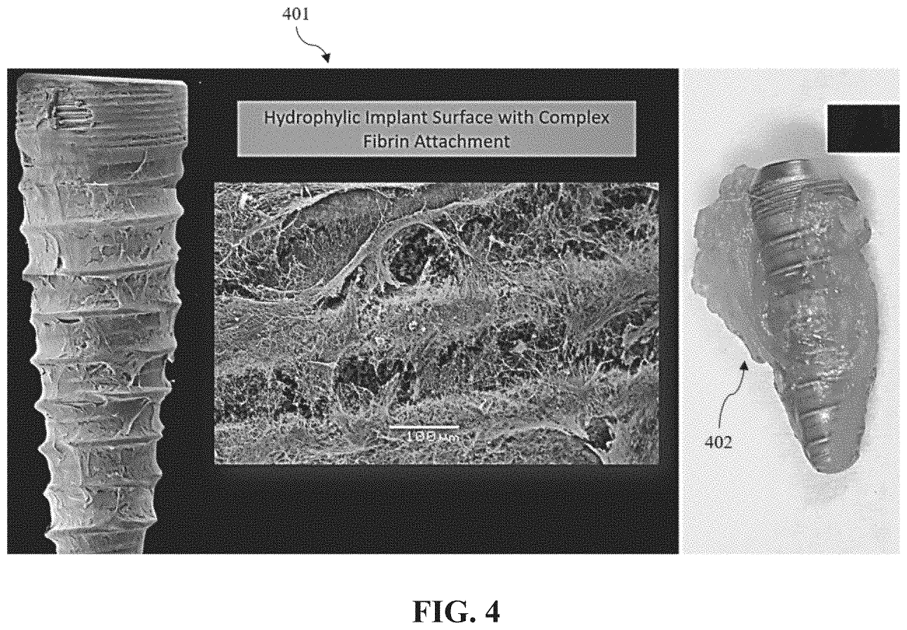

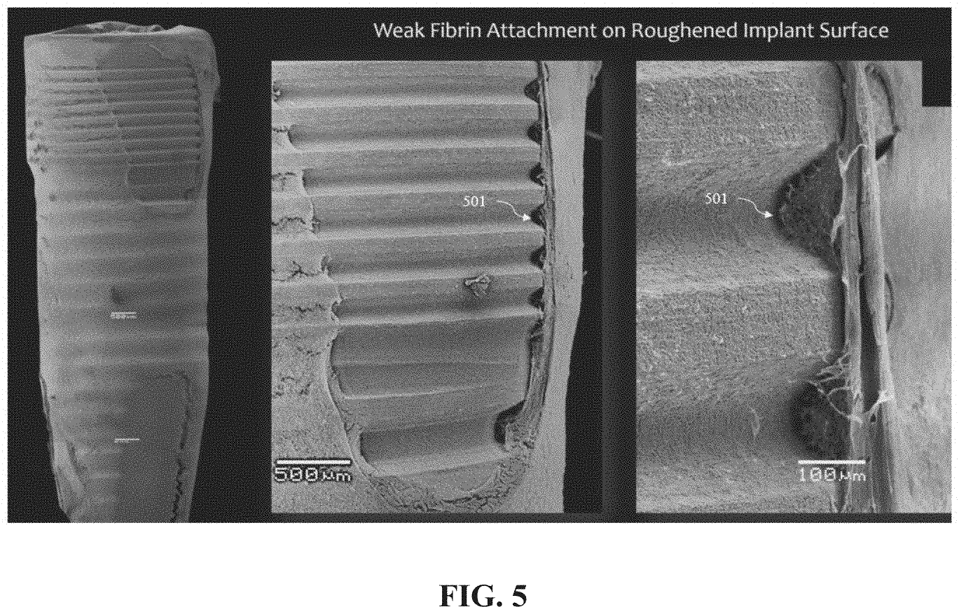

FIG. 4 illustrates the complex fibrin attachment structure 401 after a UVC treatment as described herein according to some embodiments. As shown, the attachment is very complex, and attempts to pull the fibrin off 402 resulted in shredded fibrin as opposed to it coming off in one piece as would be expected in a non-UVC treated implant according to some embodiments. FIG. 5 depicts an undesirable attachment structure for an implant not treated with UVC comprising low density adhesion and or gaps 501 according to some embodiments.

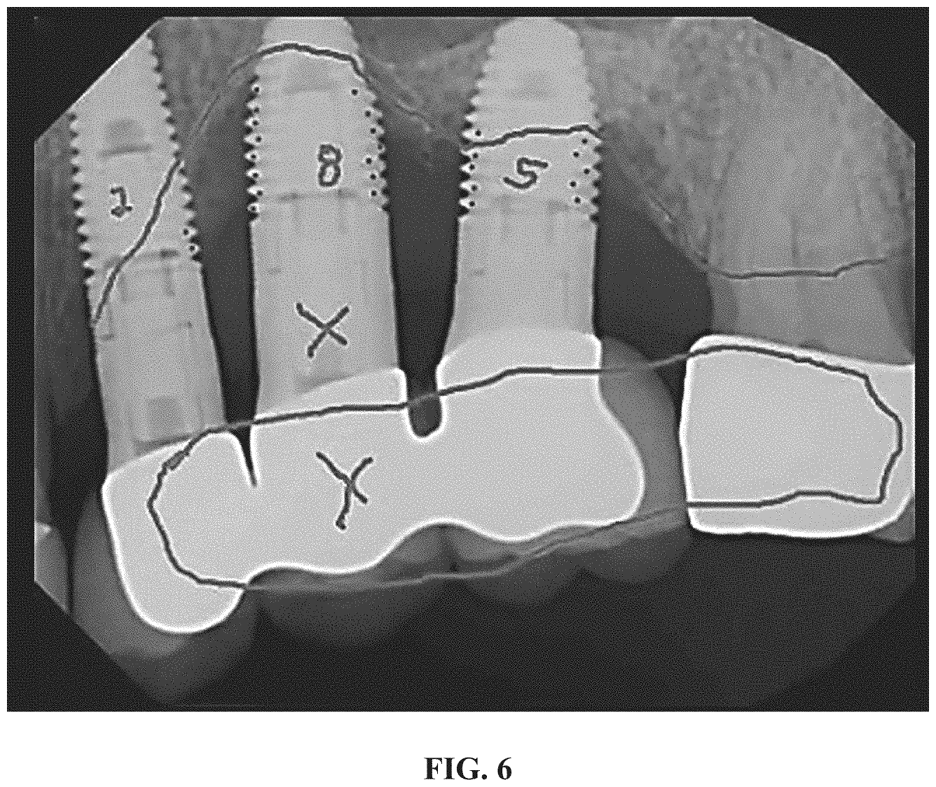

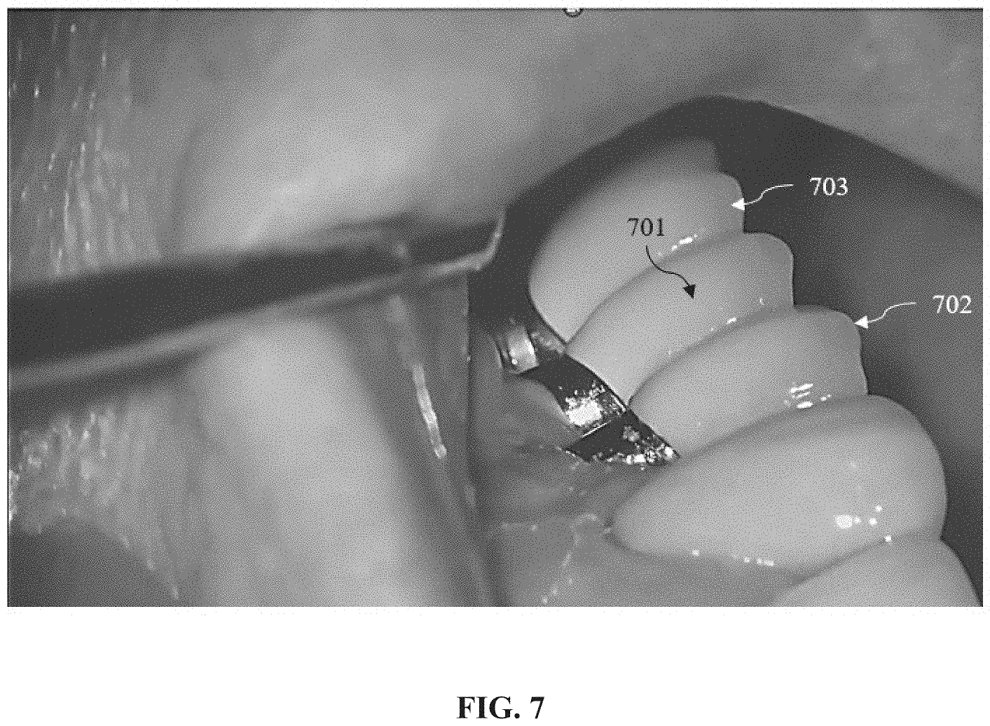

FIG. 6 is a radiograph of an example peri-implantitis site before treatment according to the methods described herein according to some embodiments. FIG. 7 is a high-resolution picture of the peri-implantitis site shown in FIG. 6 before treatment according to the methods described herein according to some embodiments. In some embodiments, the first step in the method is to remove at least a portion of the implant components 701 and prepare the site for grafting. In some embodiments, it is necessary to graft the entire area between the bone structures of the #12 implant 702 and #14 implant 703, as a non-limiting example, to create a new bone structure for the entire implant site.

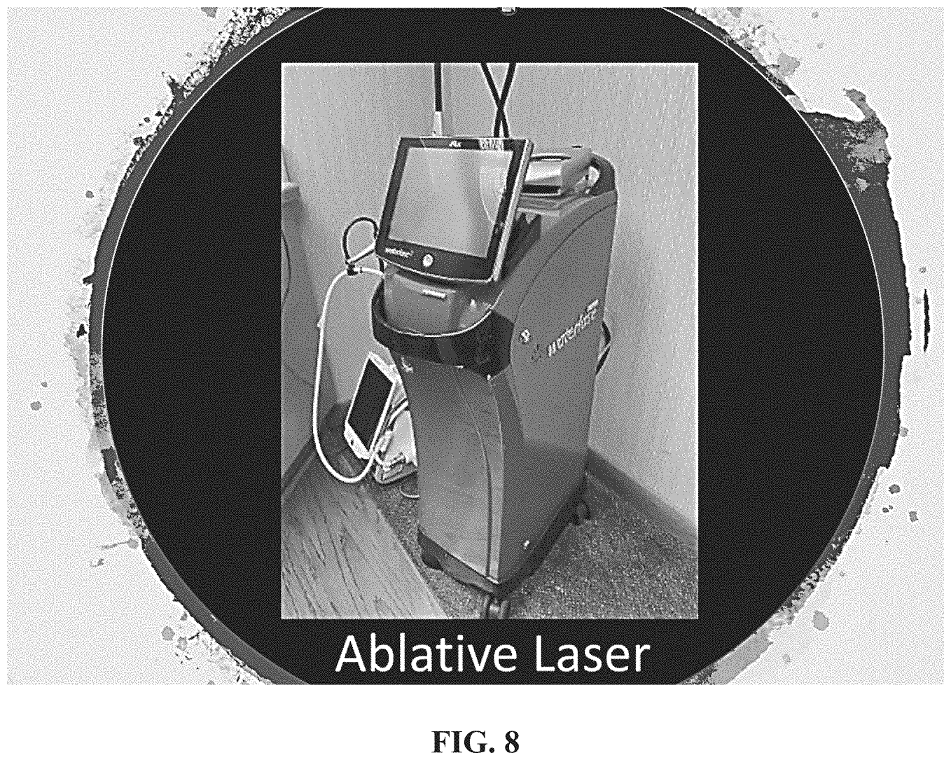

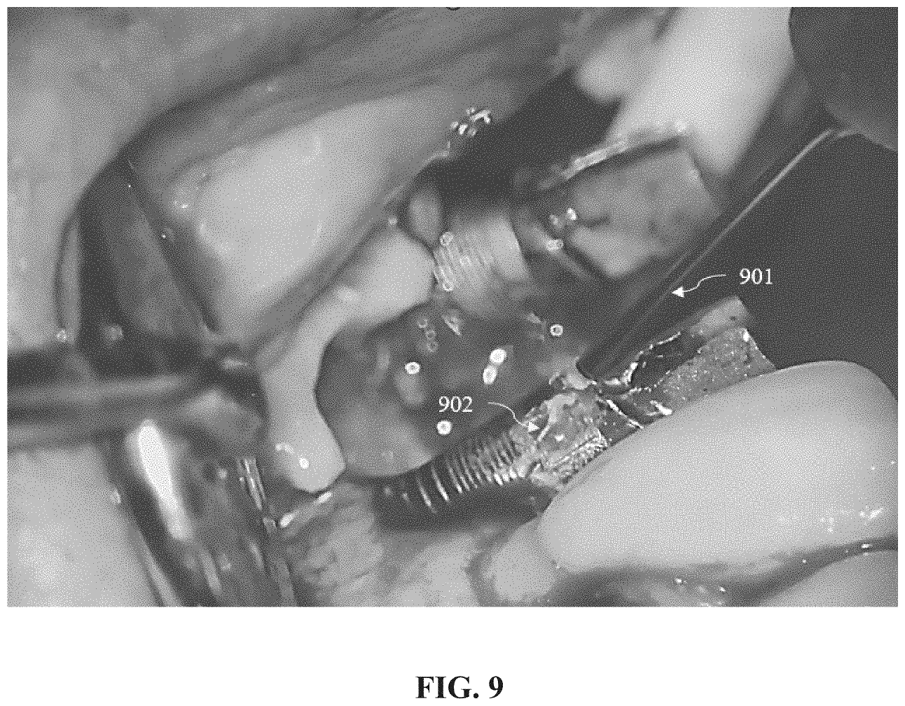

In some embodiments, the next step in the method is to clean and disinfect the surface of the implant. FIG. 8 is a picture of an ablative laser suitable for cleaning and disinfecting the surface of one or more implants according to some embodiments. FIG. 9 is a high-resolution picture showing the ablative laser 901 of FIG. 8 cleaning the exposed implant surfaces 902 according to some embodiments. In some embodiments, the ablative laser 901 is used to first remove the granulation tissue and/or pathologic tissue that is residual in the defect area. In some embodiments, the next step is to use the ablative laser 901 to clean the bioburden from the exposed implant surface including areas between the implant's threads.

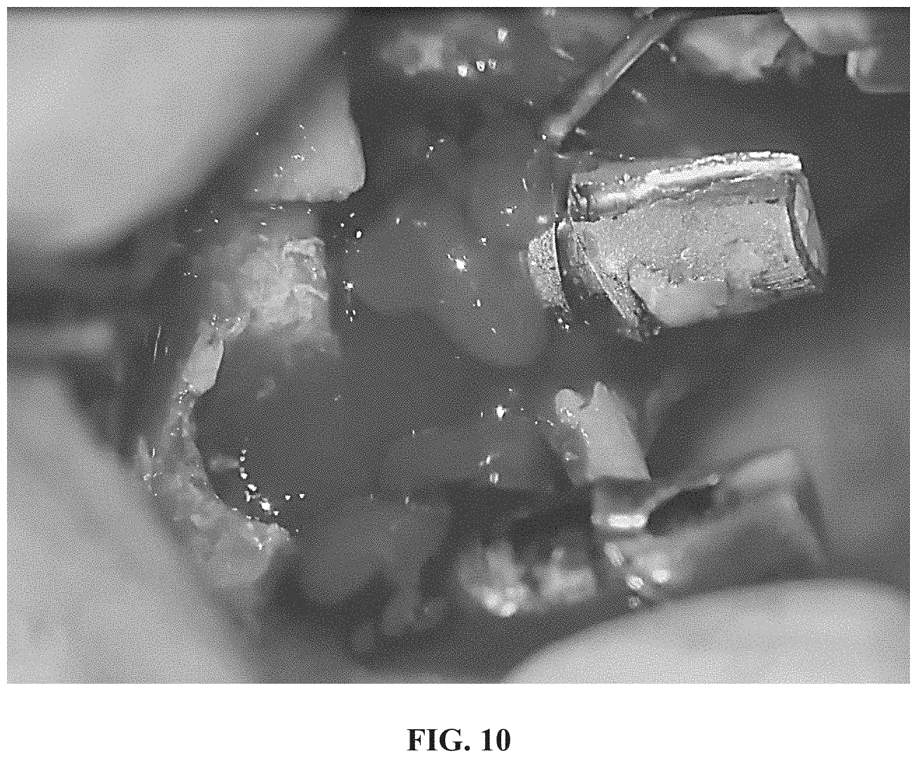

While the implant surface may be visually clean, at the atomic level, the aged titanium is still contaminated and in a hydrophobic state. It has been found that even a treatment with an ablative laser is not sufficient to remove enough of the contaminants to return the implant surface to a hydrophilic state. In some embodiments, the next step to clean the implant surface 902 and prepare it for graft material is to acid etch the exposed implant surface to remove bacterial endotoxins. FIG. 10 depicts an acid etching step in accordance with some embodiments. In some embodiments, after the acid etch is complete, the acid is removed and/or washed away. In some embodiments, the next step is to remove the denatured endotoxin. A suitable compound for this procedure is ethylenediaminetetraacetic acid (EDTA), although other substances and compounds may also be used.

In some embodiments, a subsequent step is to remove any residual acid from the implant site by flooding it with hydrogen peroxide (H₂O₂) which also removes any remaining bioburden. Although these steps contemplate a best mode, other methods can be employed to satisfy a step of cleaning and preparing the area for a treatment using UVC light with a wavelength of approximately 254 nm according to some embodiments. Some embodiments apply UVC in the range of plus or minus five nm from the 254 nm application. Some embodiments described herein include the all or part of the UVC wavelength range of (100-280 nm) and, in some embodiments, also includes the use of UVA both independently and/or in conjunction with each other.

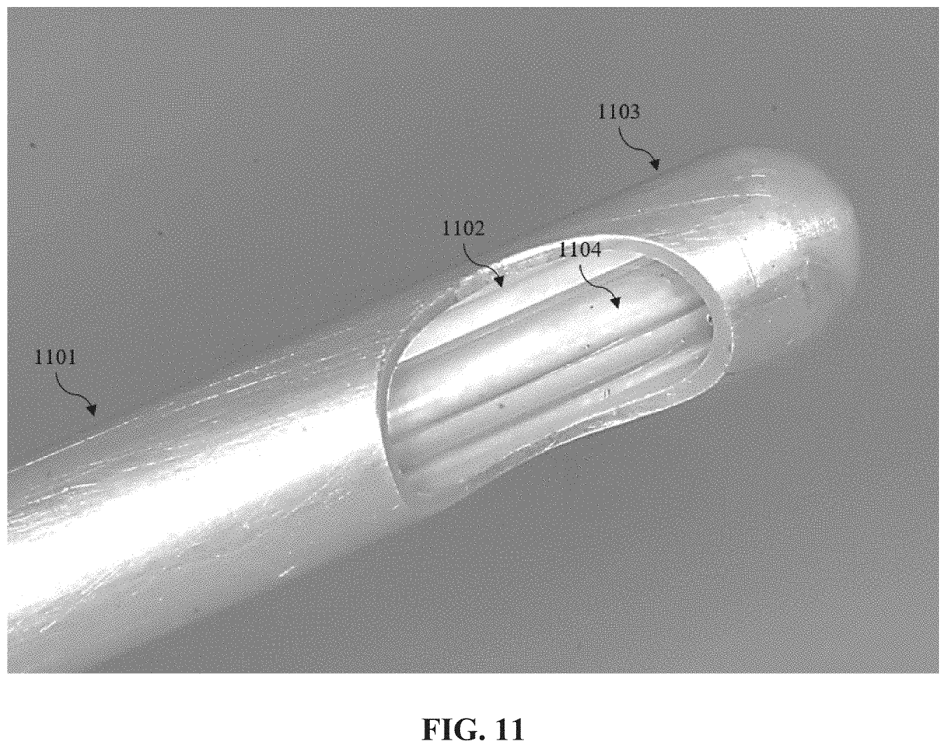

In some embodiments, after the cleaning step, a UVC treatment at 253.7 nm (as used herein, a references to 254 nm and 253.7 nm are interchangeable for the purposes of defining the metes and bounds of the claims) is applied to exposed implant surfaces. In some embodiments, a method of applying the UVC treatment includes a step applying the UVC to the implant as not to exceed an implant surface temperature of 40° C. In some embodiments, the system is configured to modulate the power of the UVC source to prevent an implant from reaching a temperature over 40° C. FIG. 11 shows a UVC instrument that is configured to direct exposure from a mercury lamp generating UVC at 254 nm to an implant surface according to some embodiments. In some embodiments, the UVC instrument includes a handpiece 1101, a side opening 1102 located proximate a distal end 1103, and a UVC light 1104. In some embodiments, the method includes a UVC treatment at approximately 254 nm (e.g., 253.7 nm) with one or more UVC instruments described herein.

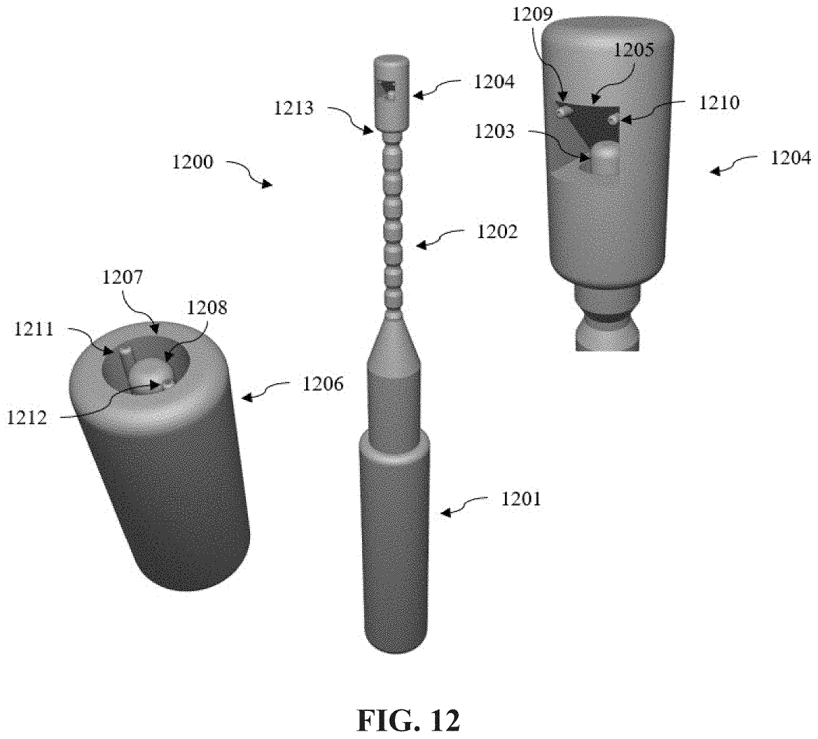

FIG. 12 illustrates a flexible handpiece 1200 for directing ultraviolet light (e.g., UVC light) onto a partially exposed implant according to some embodiments. In some embodiments, the flexible handpiece 1200 comprises a handpiece base 1201. In some embodiments, attached to the handpiece base 1201 is a flexible neck 1202. In some embodiments, the flexible neck 1202 is configured to bend and hold a bended position in order to direct light from a UVC light 1203 disposed in the tip 1204 within a patient's mouth. In some embodiments, the bending function can be provided by pivoting and/or sliding elements and/or links.

In some embodiments, the light is emitted from a side light opening 1205. In some embodiments, the tip 1204 is configured to rotate about its longitudinal axis 1213 where it connects to the flexible neck 1202. In some embodiments, the flexible neck 1202 is configured to rotate about a longitudinal axis of the handpiece base 1201, wherein the tip 1204 is configured to rotate with the flexible neck 1202. In some embodiments, the tip 1204 is removable from the flexible neck 1202 and/or handpiece base 1201. In some embodiments, the flexible neck 1202 and/or handpiece base 1201 is configured to enable multiple different types of tips, such as tip 1206. In some embodiments, the tip 1206 comprises a distal opening 1207 configured to enable light from UVC light 1208 to shine therethrough.

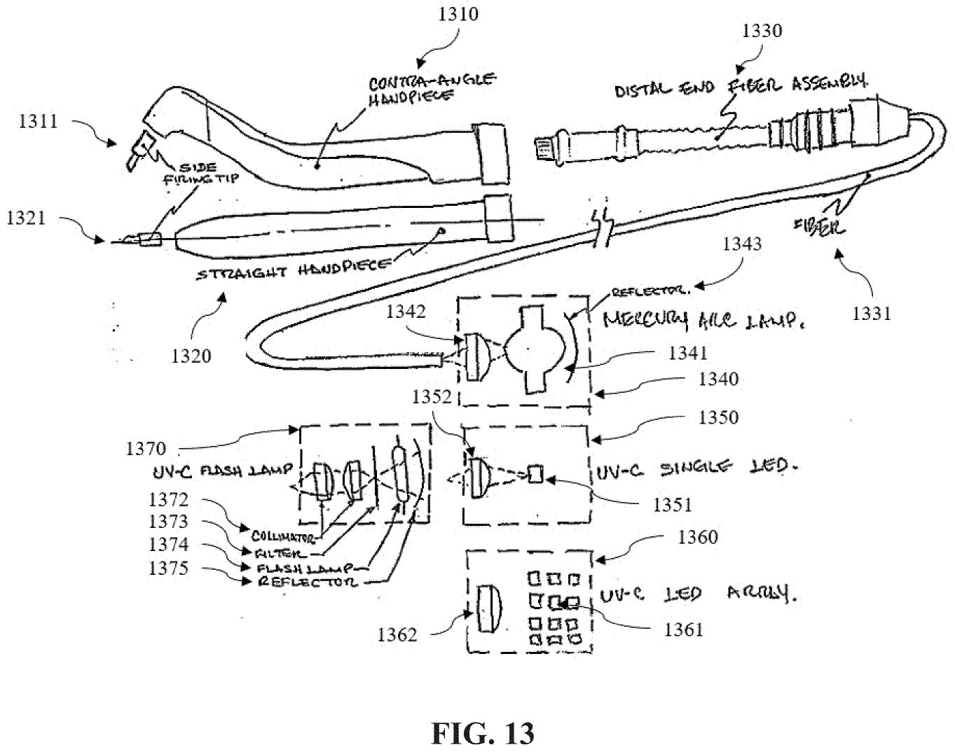

FIG. 13 illustrates two interchangeable handpiece variations and removable light transmission component according to some embodiments. In some embodiments, the handpiece includes a contra-angle handpiece 1310 comprising a side firing tip 1311 (or end firing tip). In some embodiments, the handpiece includes a straight handpiece 1320 comprising a side firing tip 1321 (or end firing tip). In some embodiments, one or more handpieces (e.g., the flexible handpiece 1200, the contra-handle handpiece 1310, the straight hand piece 1320, and/or combinations thereof) are configured to couple to a fiber assembly 1330 at a distal end thereof. In some embodiments, the fiber assembly 1330 comprises a fiber optic cable 1331 configured to transmit light from one or more light emitting systems comprising one or more of a mercury arc lamp system 1340, a single UVC LED system 1350, a UVC LED array system 1360, and a UVC flash lamp system 1370.

In some embodiments, the mercury arc lamp system 1340 includes a reflector 1343 configured to reflect light from a mercury arc lamp 1341 into a lensing system (e.g., one or more collimators) 1342 which then focuses the UVC light into the fiber optic cable 1331. In some embodiments, the fiber optic cable 1331 is configured to transmit the UVC light to the fiber assembly 1330 which is configured to direct the UVC light out of the side firing tip 1311, 1321 (or end firing tip) when the fiber assembly 1330 is inserted into one or more handpieces described herein.

In some embodiments, the single UVC LED system 1350 comprises a single UVC LED 1351. In some embodiments, the single UVC LED system 1350 is configured to direct UVC light emitted from the single UVC LED 1351 into the lensing system 1352 which focuses the light into the fiber assembly 1330 which then directs the UVC light out the side firing tip 1311, 1321 (or end firing tip).

In some embodiments, a UVC LED array system 1360 comprises a plurality of UVC LEDs 1361. In some embodiments, the UVC LED array system 1360 is configured to direct UVC light emitted from the UVC LED array 1361 into the lensing system 1362 which focuses the light into the fiber assembly 1330 and subsequently out the side firing tip 1311, 1321 (or end firing tip).

In some embodiments, the UVC flash lamp system 1370 comprises a lensing system 1372, one or more filters 1373, one or more UVC flash lamps 1374, and one or more reflectors 1375 configured to direct UVC light into the fiber assembly 1330 and subsequently out the side firing tip 1311, 1321 (or end firing tip).

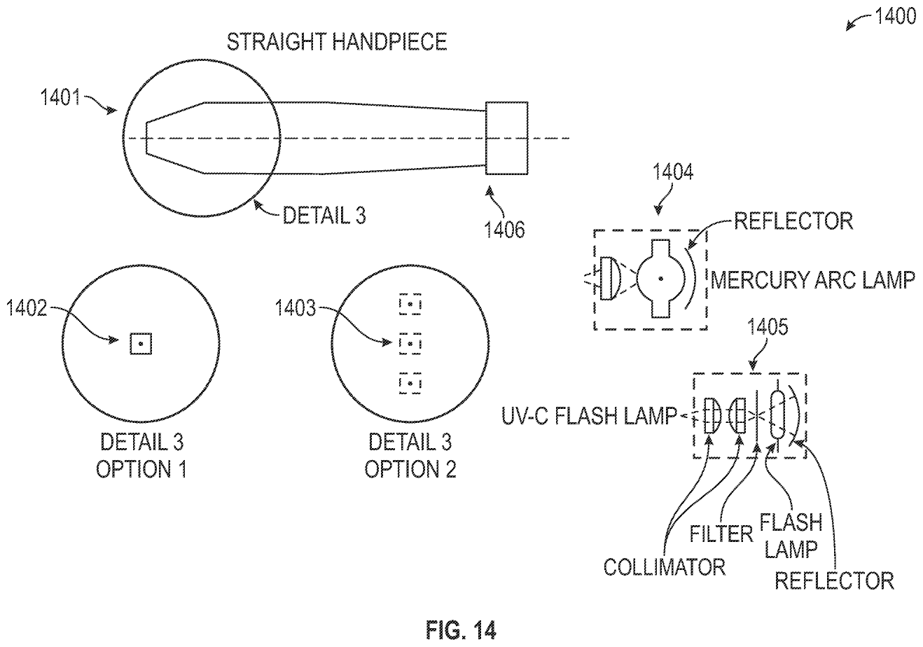

FIG. 14 depicts a straight handpiece 1400 with one or more UVC LEDs positioned at a distal end of the straight handpiece 1400 according to some embodiments. In some embodiments, the straight handpiece 1400 distal end 1401 comprises a single UVC LED 1402, a UVC LED array 1403, a mercury arc lamp system 1404, and/or a flash lamp system 1405 within a handpiece body 1406 configured to project UVC light from the distal end 1401.

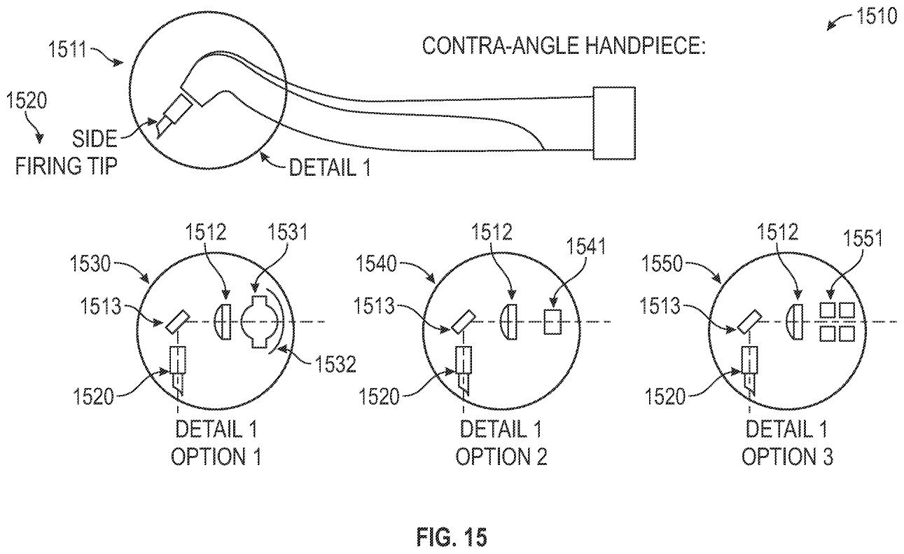

FIG. 15 shows a contra-angle handpiece 1510 and one or more UVC light projecting arrangements 1530, 1540, 1550 according to some embodiments. In some embodiments, a distal end 1511 of the contra-angle handpiece 1510 comprises one or more of a lamp 1531 (e.g., a mercury arc lamp or flash lamp configured to emit UVC light), a single UVC LED 1541, and/or a UVC LED array 1551. In some embodiments, the distal end 1511 comprises a reflector 1532. In some embodiments, the reflector 1532 is configured to reflect light from one or more UVC light sources 1531, 1541, and/or 1551 toward the lensing system 1512. In some embodiments, the lensing system 1512 at the distal end 1511 is configured to direct UVC light onto an angled mirror 1513. In some embodiments, the angled mirror 1513 is configured to direct the light to a side firing tip 1520 (or end firing tip) configured to deliver the UVC light to an implant surface. In some embodiments, with regard to any handpiece variation described herein, an end firing tip is used in place of a side firing tip (or end firing tip).

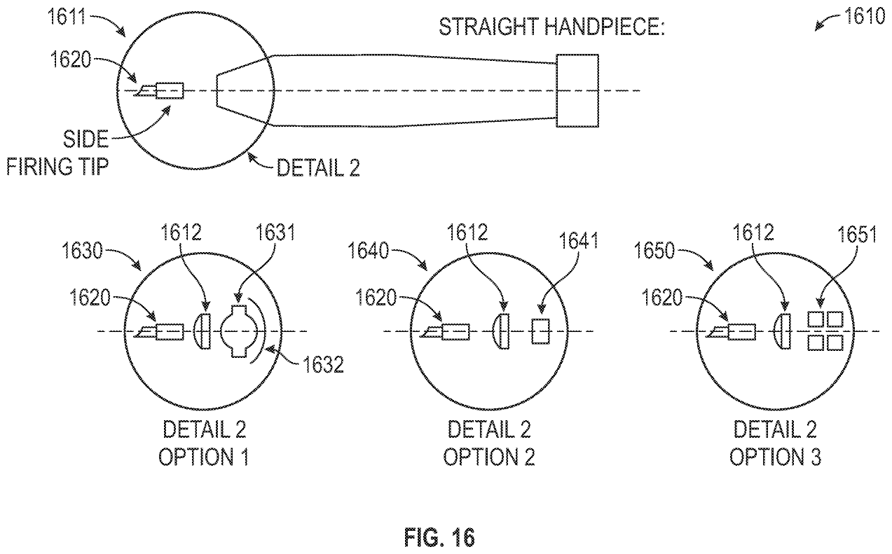

FIG. 16 shows a straight handpiece 1610 and one or more UVC light projecting arrangements 1630, 1640, 1650 according to some embodiments. In some embodiments, a distal end 1611 of the straight handpiece 1610 comprises one or more of a lamp 1631 (e.g., a mercury arc lamp or flash lamp configured to emit UVC light), a single UVC LED 1641, and/or a UVC array 1651. In some embodiments, one or more UVC light sources 1631, 1641, and/or 1651 comprise a reflector configured to reflect light to a lensing system. In some embodiments, the distal end 1611 comprises a lensing system 1612 configured to direct UVC light into an end firing tip 1620 (or an end firing tip according to some embodiments) configured to deliver the UVC light to an implant surface. In some embodiments, the straight handpiece 1610 comprises a

In some embodiments, a graft material is then selected and applied. An absorbent graft material is preferred, as the harder, more crystalline, less absorbable the graft material will reduce attachment to the conditioned titanium surface according to some embodiments. In some embodiments, xenografts, cortical bone chips, a combination of cortical and cellulose bone grafts have been found to be less effective as the hydroxyapatite (HA) density increases, the more chance of forming a multinucleated giant cell complex occurs which leads to connective tissue formation instead of bone. In some embodiments, cancellous allograt and/or alloplast that fully absorb and release free ionic calcium has given satisfactory results and can be combined with autologous biologics.

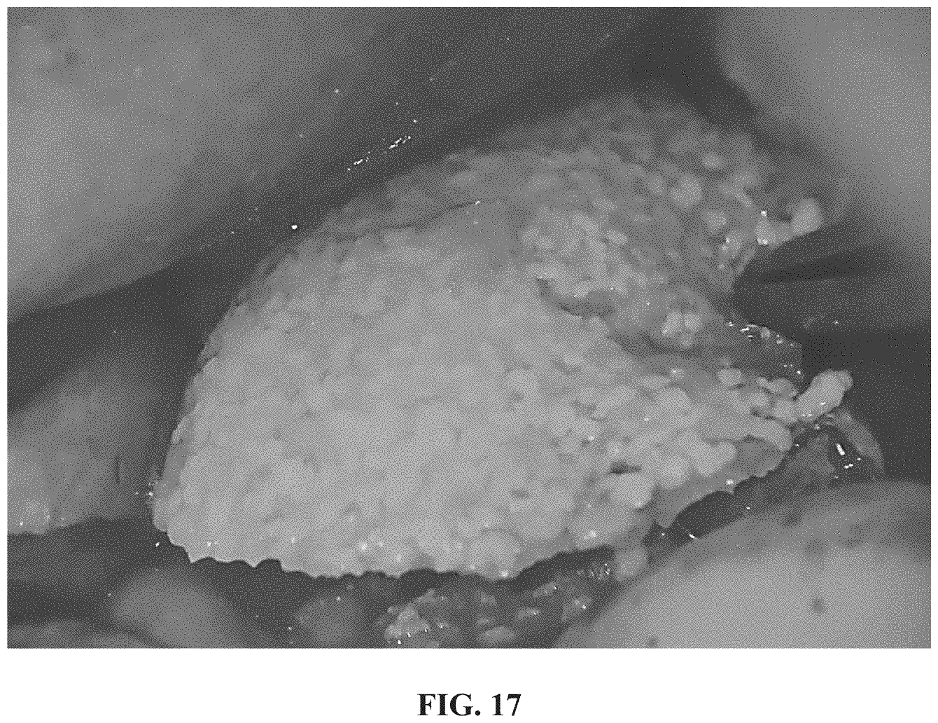

FIG. 17 shows the application of particulate that was formed in a dish according to some embodiments. Using this method, the particulates are formed around the implant bodies, which prevents the particulates from migrating out of the site according to some embodiments. In some embodiments, the particulates are condensed with some bone pluggers, and material is continued to be added until the ideal volume is reached.

In some embodiments, after the graft material is added, the next step is to add a collagen membrane barrier. In some embodiments, in many peri-implantitis cases key walls are missing from the defect site. In some embodiments, when the walls are missing only a barrier (e.g., an absorbable collagen barrier) will help to maintain the integrity of the grafting site. In some embodiments, punches are formed in the membrane (e.g., two in this case) which are configured and arranged to align with the remaining implants. The implants pass through the membrane and the membrane is draped and formed over the bone graft complex. In some embodiments, the absorbable collagen membrane should not be the last layer as it is not bioreactive.

In some embodiments, a subepithelial connective tissue graft (SECT) is used, which often comes from a palatal donor site, or if more tissue is needed, soft tissue such as alloderm. In some embodiments, the final layer is an autologous fibrin membrane in the form of L-PRF (leucocyte containing platelet rich fibrin). In some embodiments, buttonholes (e.g., two small buttonholes) are made in the PRFmembrane, which allow the membrane to stretch over and between the two implant abutments and the membrane is then pushed down to the shoulder of the two abutments. In some embodiments, this allows for good closure without having to worry about getting perfect primary flap closure over the graft material. In some embodiments, a temporary bridge is then placed over the final layer. In some embodiments, a periosteal release can also be used to get good primary closure over the collagen membrane and the PRFmembrane.

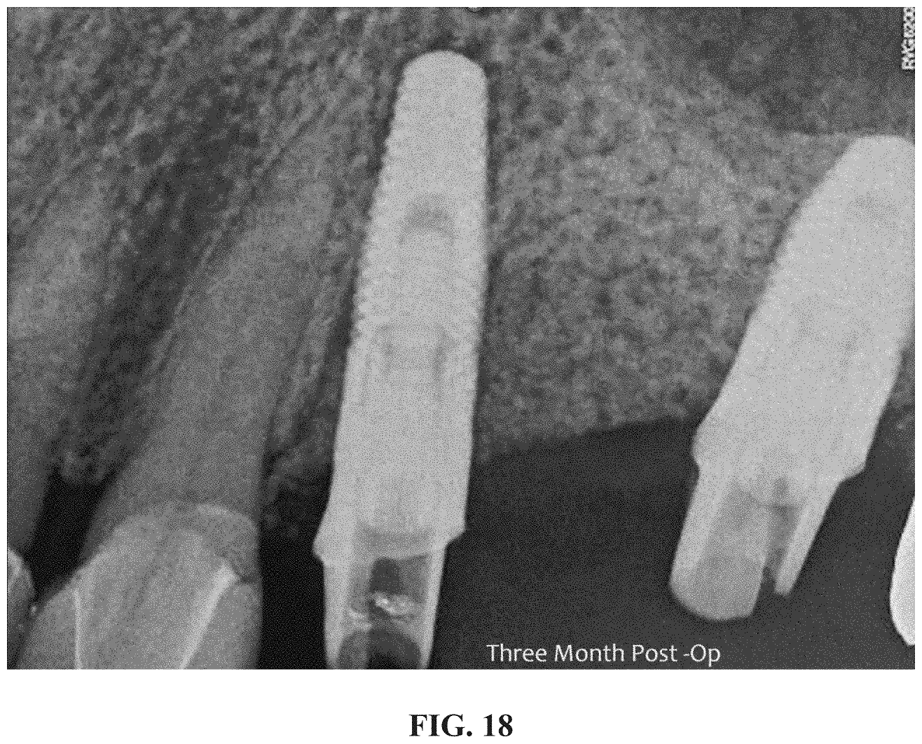

FIG. 18 shows a 3-month post op radiograph of the same case according to some embodiments. In some embodiments, this radial graft volume is typical of the results seen with UVC treatment of an exposed implant surface according to the method described herein. In some embodiments, the radiograph shows strong adhesion to previously exposed implant surface as well as new bone growth in the graft area, allowing for a new implant at the same site. In some embodiments, both the new and used implants are exposed to UVC light using the UVC instrument described herein. In some embodiments, lab milled posts are also treated with the UVC described herein for soft tissue adhesion.

In some embodiments, the disclosure is also directed to a UVC instrument configured and arranged to deliver UVC light at 254 nm to the exposed implant surfaces. As used herein, an exposed implant surface is any portion of an implant of any material that is surgically secured within the mouth of a patient, and where at least a portion of the implant is not covered by bone and/or tissue. Some embodiments of the system are directed to a UVC instrument configured and arranged to apply UVC light at 254 nm to a posterior portion of an exposed implant surface, which may be facing the posterior portions of the oral cavity and/or away from the mouth and/or not visible when looking through the mouth.

In some embodiments, the UVC instrument including handpiece 1200 comprises one or more fluid tubes 1209, 1210, 1211, and 1212. In some embodiments, at least one of the one or more fluid tubes 1209-1212 is a fluid delivery tube configured and arranged to deliver a fluid to the implant site. In some embodiments, the fluid includes water. In some embodiments, the fluid includes an acid etch. In some embodiments, the fluid includes hydrogen peroxide. In some embodiments, the fluid is a gas. In some embodiments, the fluid is air. In some embodiments, at least one of the one or more fluid tubes is a liquid delivery tube 1209, 1211. In some embodiments, at least one of the one or more fluid tubes is a gas delivery tube 1210, 1212. In some embodiments, the UVC instrument that includes handpiece 1200 is configured to deliver both a gas and a liquid to the implant site simultaneously. One or more handpiece variations shown in FIGS. 12-16 comprise one or more tubes and/or associated functionality described herein according to some embodiments. It is understood that various features from some embodiments, such as tube arrangements, are non-limiting examples meant to aid those of ordinary skill with making and using the system. Features according to some embodiments are readily combinable with features from some other embodiments, and such interchangeability can be combinable for the purposes of defining the metes and bounds of embodiments of the system.

In some embodiments, at least one of the one or more fluid tubes 1209-1212 are fluid cooling tubes configured to remove heat from the UVC instrument. In some embodiments, the one or more fluid tubes are configured and arranged to circulate cooling fluid into, within, and/or out of a UVC tip. In some embodiments, at least a portion of the UVC instrument adjacent to or comprising the UVC tip is a neck 1202 that is flexible and/or adjustable. In some embodiments, the neck 1202 can bend up to 180° relative to the handpiece such that UVC light can be directed onto an implant surface facing the posterior portions of the oral cavity while the instrument handpiece extends outside the patient's mouth.

In some embodiments, the UVC instrument 1200 includes one or more UVC tips 1204, 1206. In some embodiments, at least a portion of the one or more UVC tips 1204, 1206 include a light opening 1205, 1207 configured and arranged to allow UVC light to pass therethrough. In some embodiments, the light opening 1207 is at distal end of the UVC tip and is substantially perpendicular to the neck. In some embodiments, the light opening 1205 is located on a side of the one or more UVC tips 1204 adjacent and/or perpendicular to a plane along a distal end of the tip 1204 and parallel to a center axis of the handpiece base 1201 and the neck 1202. In some embodiments, at least a portion of the one or more UVC tips 1204 that include a UVC light opening 1205 on the side is rotatable such that the UVC light can be directed when in a patient's mouth at any angle from 0° to 360°. In some embodiments, the system includes an ergonomic handpiece which can be coupled to a wide variety of tips. Some embodiments include a handpiece configured to be coupled to a side firing tip or an end firing tip each configured to provide a light pattern for the methods described herein. In some embodiments, the pattern is configured to keep a temperature of the implant below 40° C. In some embodiments, a distal end fiber assembly is coupled by an optical fiber to a UVC light source. In some embodiments, the UVC light source is modular and readily coupled and decoupled from the fiber. Some embodiments include a light source comprising a UVC single LED, a UVC LED array and/or a mercury arc lamp.

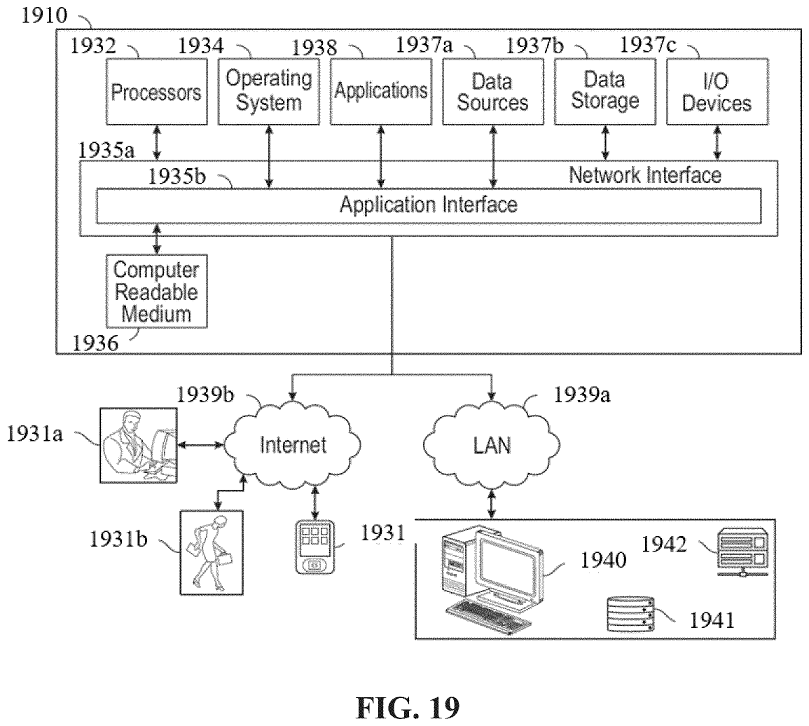

Some embodiments include a controller for the UVC instrument which can comprise any type of computer system. In some embodiments, the controller is configured to prevent the surface of the implant from reaching a temperature greater than 40° C. FIG. 19 illustrates a computer system 1910 enabling or comprising the systems and methods in accordance with some embodiments of the system. In some embodiments, the computer system 1910 can operate and/or process computer-executable code of one or more software modules of the aforementioned system and method. Further, in some embodiments, the computer system 1910 can operate and/or display information within one or more graphical user interfaces (e.g., HMIs) integrated with or coupled to the system.

In some embodiments, the computer system 1910 can comprise at least one processor 1932. In some embodiments, the at least one processor 1932 can reside in, or coupled to, one or more conventional server platforms (not shown). In some embodiments, the computer system 1910 can include a network interface 1935a and an application interface 1935b coupled to the least one processor 1932 capable of processing at least one operating system 1934. Further, in some embodiments, the interfaces 1935a, 1935b coupled to at least one processor 1932 can be configured to process one or more of the software modules (e.g., such as enterprise applications 1938). In some embodiments, the software application modules 1938 can include server-based software, and can operate to host at least one user account and/or at least one client account, and operate to transfer data between one or more of these accounts using the at least one processor 1932.

With the above embodiments in mind, it is understood that the system can employ various computer-implemented operations involving data stored in computer systems. Moreover, the above-described databases and models described throughout this disclosure can store analytical models and other data on computer-readable storage media within the computer system 1910 and on computer-readable storage media coupled to the computer system 1910 according to various embodiments. In addition, in some embodiments, the above-described applications of the system can be stored on computer-readable storage media within the computer system 1910 and on computer-readable storage media coupled to the computer system 1910. In some embodiments, these operations are those requiring physical manipulation of physical quantities. Usually, though not necessarily, in some embodiments these quantities take the form of one or more of electrical, electromagnetic, magnetic, optical, or magneto-optical signals capable of being stored, transferred, combined, compared and otherwise manipulated. In some embodiments, the computer system 1910 can comprise at least one computer readable medium 1936 coupled to at least one of at least one data source 1937a, at least one data storage 1937b, and/or at least one input/output 1937c. In some embodiments, the computer system 1910 can be embodied as computer readable code on a computer readable medium 1936. In some embodiments, the computer readable medium 1936 can be any data storage that can store data, which can thereafter be read by a computer (such as computer 1940). In some embodiments, the computer readable medium 1936 can be any physical or material medium that can be used to tangibly store the desired information or data or instructions and which can be accessed by a computer 1940 or processor 1932. In some embodiments, the computer readable medium 1936 can include hard drives, network attached storage (NAS), read-only memory, random-access memory, FLASH based memory, CD-ROMs, CD-Rs, CD-RWs, DVDs, magnetic tapes, other optical and non-optical data storage. In some embodiments, various other forms of computer-readable media 1936 can transmit or carry instructions to a remote computer 1940 and/or at least one user 1931, including a router, private or public network, or other transmission or channel, both wired and wireless. In some embodiments, the software application modules 1938 can be configured to send and receive data from a database (e.g., from a computer readable medium 1936 including data sources 1937a and data storage 1937b that can comprise a database), and data can be received by the software application modules 1938 from at least one other source. In some embodiments, at least one of the software application modules 1938 can be configured within the computer system 1910 to output data to at least one user 1931 via at least one graphical user interface rendered on at least one digital display.

In some embodiments, the computer readable medium 1936 can be distributed over a conventional computer network via the network interface 1935a where the system embodied by the computer readable code can be stored and executed in a distributed fashion. For example, in some embodiments, one or more components of the computer system 1910 can be coupled to send and/or receive data through a local area network (“LAN”) 1939a and/or an internet coupled network 1939b (e.g., such as a wireless internet). In some embodiments, the networks 1939a, 1939b can include wide area networks (“WAN”), direct connections (e.g., through a universal serial bus port), or other forms of computer-readable media 1936, or any combination thereof.

In some embodiments, components of the networks 1939a, 1939b can include any number of personal computers 1940 which include for example desktop computers, and/or laptop computers, or any fixed, generally non-mobile internet appliances coupled through the LAN 1939a. For example, some embodiments include one or more of personal computers 1940, databases 1941, and/or servers 1942 coupled through the LAN 1939a that can be configured for any type of user including an administrator. Some embodiments can include one or more personal computers 1940 coupled through network 1939b. In some embodiments, one or more components of the computer system 1910 can be coupled to send or receive data through an internet network (e.g., such as network 1939b). For example, some embodiments include at least one user 1931a, 1931b, is coupled wirelessly and accessing one or more software modules of the system including at least one enterprise application 1938 via an input and output (“I/O”) 1937c. In some embodiments, the computer system 1910 can enable at least one user 1931a, 1931b, to be coupled to access enterprise applications 1938 via an I/O 1937c through LAN 1939a. In some embodiments, the user 1931 can comprise a user 1931a coupled to the computer system 1910 using a desktop computer, and/or laptop computers, or any fixed, generally non-mobile internet appliances coupled through the internet 1939b. In some embodiments, the user can comprise a mobile user 1931b coupled to the computer system 1910. In some embodiments, the user 1931b can connect using any mobile computing 1931c to wireless coupled to the computer system 1910, including, but not limited to, one or more personal digital assistants, at least one cellular phone, at least one mobile phone, at least one smart phone, at least one pager, at least one digital tablets, and/or at least one fixed or mobile internet appliances.

The subject matter described herein are directed to technological improvements to the treatment and prevention of peri-implantitis by in-situ treatment of exposed implant surfaces. The disclosure describes the specifics of how a machine including one or more computers comprising one or more processors and one or more non-transitory computer implement some embodiments of the system and its improvements over the prior art. The instructions executed by the machine cannot be performed in the human mind or derived by a human using a pen and paper but require the machine to convert process input data to useful output data. Moreover, the claims presented herein do not attempt to tie-up a judicial exception with known conventional steps implemented by a general-purpose computer; nor do they attempt to tie-up a judicial exception by simply linking it to a technological field. Indeed, the systems and methods described herein were unknown and/or not present in the public domain at the time of filing, and they provide a technologic improvements advantages not known in the prior art. Furthermore, the system includes unconventional steps that confine the claim to a useful application.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Furthermore, acting as Applicant's own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C, or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured unless otherwise specified.

“Simultaneously” as used herein includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry. For recited physical steps, “simultaneously” includes a time difference between steps up to 5 seconds.

As used herein, “can” or “may” or derivations there of (e.g., the system display can show X) are used for descriptive purposes only and is understood to be synonymous and/or interchangeable with “configured to” (e.g., the computer is configured to execute instructions X) when defining the metes and bounds of the system.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited. Another example is “a computer system configured to or programmed to execute a series of instructions X, Y, and Z.” In this example, the instructions must be present on a non-transitory computer readable medium such that the computer system is “configured to” and/or “programmed to” execute the recited instructions: “configure to” and/or “programmed to” excludes art teaching computer systems with non-transitory computer readable media merely “capable of” having the recited instructions stored thereon but have no teachings of the instructions X, Y, and Z programmed and stored thereon. The recitation “configured to” can also be interpreted as synonymous with operatively connected when used in conjunction with physical structures.

The previous detailed description is to be read with reference to the figures, in which like elements in different figures have like reference numerals. The figures, which are not necessarily to scale, depict some embodiments and are not intended to limit the scope of embodiments of the system.

Any of the operations described herein that form part of the invention are useful machine operations. The invention also relates to a device or an apparatus for performing these operations. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g. a cloud of computing resources.

The embodiments of the invention can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data.

Although method operations are presented in a specific order according to some embodiments, the execution of those steps do not necessarily occur in the order listed unless explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

We claim:

1. A method for treating peri-implantitis of one or more dental implants embedded in a patient's mouth using ultraviolet light, the method including steps of:
(1) cleaning an area around the one or more dental implants;
(2) preparing the area for graft material;
(3) removing remaining bioburden from an exposed implant surface;
(4) applying an ultraviolet light treatment to the exposed implant surface;
(5) applying a graft material around the exposed implant surface; and
(6) applying a collagen barrier over the graft material.

2. The method of claim 1,
wherein the step of cleaning the area comprises removing granulation tissue and/or pathologic tissue from around the one or more dental implants.

3. The method of claim 1,
wherein the step of removing remaining bioburden comprises treating the exposed implant surface with directed laser energy.

5. The method of claim 1,
wherein the step of providing the ultraviolet light treatment to the exposed implant surface includes providing ultraviolet c (UVC) light to the exposed implant surface at a wavelength range of 100 nm to 280 nm.

6. The method of claim 1,
wherein the step of providing the ultraviolet light treatment to the exposed implant surface includes providing ultraviolet c (UVC) light to the exposed implant surface at a wavelength range of 249 nm to 259 nm.

7. The method of claim 1,
wherein the step of providing the ultraviolet light treatment to the exposed implant surface includes providing an ultraviolet light instrument configured to apply ultraviolet c (UVC) light to the exposed implant surface at a wavelength range of 100 nm to 280 nm.

8. The method of claim 7,
wherein the ultraviolet light instrument is configured to apply the UVC light to the exposed implant surface, the exposed implant surface at least partially facing a posterior portion and/or a medial portion of an oral cavity of the patient.

9. The method of claim 7,
wherein the ultraviolet light instrument comprises a straight dental handpiece.

10. The method of claim 9,
wherein the collagen barrier is configured to secure the graft material substantially in place for healing and restoration.

11. A apparatus for treating implants embedded in a patient's mouth comprising:
a handpiece,
an ultraviolet light source, and
a tip;
wherein the tip is coupled to a distal end of the handpiece;
wherein the handpiece is configured to direct ultraviolet light from the ultraviolet light source to the tip; and
wherein the handpiece and the tip are configured to enable the ultraviolet light to be directed onto an exposed surface of a dental implant within the patient's mouth.

12. The apparatus of claim 11,
wherein at least a portion of the exposed surface is facing a posterior portion of an oral cavity.

13. The apparatus of claim 11,
wherein at least a portion of the exposed surface is not visible when looking into the patient's mouth.

14. The apparatus of claim 11,
wherein the handpiece and the tip are configured to enable the ultraviolet light to be directed onto the exposed surface of the dental implant while at least a portion of the handpiece extends outside the patient's mouth.

15. The apparatus of claim 11,
wherein the handpiece comprises a contra angle dental handpiece;
wherein the tip is one of an end tip and a side firing tip;
wherein the contra angle dental handpiece is configured to couple to the side firing tip or the end firing tip; and
wherein the side firing tip and the end firing tip are each configured to direct the ultraviolet light onto the exposed surface of the dental implant.

16. The apparatus of claim 11,
wherein the handpiece further comprises a flexible neck;
wherein the flexible neck is configured to bend to direct the ultraviolet light onto the exposed surface.

17. The apparatus of claim 16,
wherein the tip is configured to be coupled to the flexible neck.

18. The apparatus of claim 17,
wherein the tip is one of an end tip and a side firing tip.

19. The apparatus of claim 17,
wherein the tip is configured to emit the ultraviolet light from a side surface of the tip.

20. The apparatus of claim 18,
wherein the ultraviolet light is ultraviolet c light (UVC) with a wavelength between 100-280 nm.
